(19) United States
(12) Patent Application Publication
(10) Pub. No.: US 2026/0306082 A1
(43) Pub. Date: Oct. 8, 2026

(54) Four-Degrees-of-Freedom Interventional Handle Component

(71) Applicant: Shenzhen Cardioacc Ltd, Guangdong (CN)

(72) Inventors: Xinhuan Zhou, Guangdong (CN); Jun Ma, Guangdong (CN); Jiahui Zhao, Guangdong (CN); Yuqin Liu, Guangdong (CN)

(21) Appl. No.: 18/728,130

(22) PCT Filed: Jan. 15, 2024
(86) PCT No.: PCT/CN2024/072243

§ 371 Date: Jun. 29, 2026

(30) Foreign Application Priority Data
Apr. 13, 2023 (CN) 202320900899.7

(51) Int. Cl. A61B 17/00; A61M 25/01

(52) U.S. Cl. CPC A61B 17/00234; A61B 2017/00327; A61M 25/0136; A61M 25/0147

(57) ABSTRACT

Provided by the present disclosure is an interventional handle component with four degrees of freedom, including an operating handle catheter component, and a connector component. The operating handle includes a holding housing, a transmission module is accommodated in the holding housing, and at least four traction wires are arranged in the catheter component. The traction wire is fixedly connected to a front end of the catheter component, and a rear end of the traction wire is connected to the transmission module. The transmission module includes a first transmission component for controlling two of the traction wires to bend in a first plane, and a second transmission component for controlling the other two traction wires to bend in a second plane. A front end, located at the holding housing, of the operating handle is provided with a front thumbwheel and a rear thumbwheel.

This patent application claims the priority of Chinese Patent Application No. CN202320900899.7 filed with the China National Intellectual Property Administration on Apr. 13, 2023, and entitled “Interventional handle component with four degrees of freedom”, the entire content of which is incorporated by reference in the present application.

### TECHNICAL FIELD

The present disclosure relates to the technical field of medical devices, and in particular to an interventional handle component with four degrees of freedom.

### BACKGROUND TECHNOLOGY

Minimally invasive interventional procedure is the trend of clinical development in the diagnosis and treatment of cardiovascular, respiratory, and digestive diseases, which has become the consensus and first-line diagnosis and treatment plan in China and abroad due to its high efficiency and little trauma to patients. Minimally invasive interventional procedure needs an interventional catheter for operation, and through complicated operations, such as operating the handle, and bending the front end of the catheter, a series of operations in the body of a patient can be realized.

However, the reliability of catheter interventional devices is high, thus reducing the risk to patients. In the prior art, the catheter interventional device usually employs a control structure with two degrees of freedom, but the two degrees of freedom cannot meet the existing surgical requirements. If a control structure with four degrees of freedom is provided, the manufacturing cost will be high due to the complicated structure, so a handle structure with low manufacturing cost, high reliability and lightness is urgently needed.

### SUMMARY

For the disadvantages in the prior art, an interventional handle component with four degrees of freedom is provided, including an operating handle, a catheter component connected to a front end of the operating handle, and a connector component connected to a rear end of the operating handle. The operating handle includes a holding housing, a transmission module is accommodated in an inner cavity of the holding housing, and at least four traction wires which are located at different circumferential positions and extend in an axial length are arranged in the catheter component. A front end of each traction wire is fixedly connected to a front end of the catheter component, and a rear end of the traction wire is connected to the transmission module. The transmission module includes a first transmission component for controlling two of the traction wires to bend in a first plane, and a second transmission component for controlling the other two traction wires to bend in a second plane. A front end, located at the holding housing, of the operating handle is provided with a front thumbwheel and a rear thumbwheel, the front thumbwheel is in transmission connection with the first transmission component, and the rear thumbwheel is in transmission connection with the second transmission component.

In one embodiment, the catheter component includes an outer sheath, an etching tube, and a TIP head. The etching tube is arranged in an inner cavity of the outer sheath, an outer wall of the etching tube is provided with at least four channels for the traction wires to pass through, a pull ring is fixedly embedded in a front end of the outer sheath, and each traction wire is fixedly connected to the pull ring. The TIP head is fixedly arranged at the front end of the outer sheath.

In one embodiment, the catheter component further includes a sensor. The sensor is embedded in the TIP head, and a rear end connecting line passes through the inner cavity of the outer sheath and extends to be connected to the connector component.

In one embodiment, the first transmission component includes a rotating shaft, and a slider. The rotating shaft is fixedly connected to the front thumbwheel, and the rotating shaft is threaded to the slider. The slider includes a front upper slider and a front lower slider which are separated, and two traction wires located in the first plane are fixedly connected to the front upper slider and the front lower slider, respectively. When the front thumbwheel drives the rotating shaft to rotate, the front upper slider and the front lower slider are driven to move in different front and rear directions, respectively.

In one embodiment, the first transmission component further includes a catheter fixing member, and a guide block. A front end of the catheter fixing member is used to fixedly connect the catheter component, and the catheter fixing member is installed at an inner cavity of the rotating shaft. The guide block is installed at the inner cavity of the rotating shaft, and an inner cavity of the guide cavity is provided with internal threads.

In one embodiment, surface threads of the front upper slider and the front lower slider have opposite helical directions.

In one embodiment, the second transmission component includes a support member, the surface of the support member is provided with a chute, and a rear upper slider and a rear lower slider are arranged in the chute. The rear thumbwheel is sleeved on the rear upper slider and the rear lower slider, and surfaces of the rear upper slider and the rear lower slider are provided with threads in opposite directions. When the rear thumbwheel rotates, the rear upper slider and the rear lower slider move towards opposite directions.

In one embodiment, the rear upper slider and the rear lower slider are provided with fixing blocks, and the traction wires located in the second plane are connected to the fixing blocks.

In one embodiment, the first plane and the second plane are two planes perpendicular to each other.

In one embodiment, the front and rear thumbwheels can rotate at an angle of 360°, and surfaces of the front and rear thumbwheels are provided with indication marks for indicating the rotation direction.

### THE BENEFICIAL EFFECTS OF THE INVENTION

### Beneficial Effects

The present disclosure has the beneficial effects that compared with the prior art, an interventional handle component with four degrees of freedom is provided, including an operating handle, a catheter component connected to a front end of the operating handle, and a connector component connected to a rear end of the operating handle. The operating handle includes a holding housing, a transmission module is accommodated in an inner cavity of the holding housing, and at least four traction wires which are located at different circumferential positions and extend in an axial length are arranged in the catheter component. A front end of each traction wire is fixedly connected to a front end of the catheter component, and a rear end of the traction wire is connected to the transmission module. The transmission module includes a first transmission component for controlling two of the traction wires to bend in a first plane, and a second transmission component for controlling the other two traction wires to bend in a second plane. A front end, located at the holding housing, of the operating handle is provided with a front thumbwheel and a rear thumbwheel, the front thumbwheel is in transmission connection with the first transmission component, and the rear thumbwheel is in transmission connection with the second transmission component. By providing a bendable intracardiac super catheter with four degrees of freedom and an operating handle, the scheme has low manufacturing cost, high reliability, and light device. More flexible catheter operation can be achieved, and stability is ensured, thus improving the flexibility and stability of an interventional catheter operation.

### BRIEF DESCRIPTION OF THE DRAWINGS

FIG. 1 is a three-dimensional diagram of the present disclosure;

FIG. 2 is an exploded view of a catheter component according to the present disclosure;

FIG. 3 is an exploded view of an operating handle according to the present disclosure;

FIG. 4 is an internal view of a first transmission component according to the present disclosure;

FIG. 5 is an exploded view of a first transmission component according to the present disclosure;

FIG. 6 is schematic diagram of the control on a catheter component by a first transmission component according to the present disclosure;

FIG. 7 is a three-dimensional diagram of a second transmission component according to the present disclosure;

FIG. 8 is a partial enlarged view of position A in FIG. 7 according to the present disclosure;

FIG. 9 is an exploded view of a second transmission component according to the present disclosure.

### DETAILED DESCRIPTION OF THE EMBODIMENTS

To express the present disclosure more clearly, the present disclosure is further described below with reference to accompanying drawings.

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the scope of protection of the present disclosure.

In the description of the present disclosure, it should be understood that the orientation or positional relationship indicated by terms “center”, “longitudinal”, “transverse”, “length”, “width”, “thickness”, “upper”, “lower”, “front”, “back”, “left”, “right”, “vertical”, “horizontal”, “top”, “bottom”, “inside” and “outside” is based on the orientation or positional relationship shown in the drawings only for convenience of description of the present disclosure and simplification of description rather than indicating or implying that the device or element referred to must have a particular orientation, be constructed and operate in a particular orientation, and thus are not to be construed as limiting the present disclosure. Furthermore, the terms “first” and “second” are used for descriptive purposes only and are not to be construed as indicating or implying relative importance or implying the number of the indicated technical features. Thus, the feature defined by “first” and “second” can explicitly or implicitly include one or more features. In the description of the present disclosure, “a plurality of” means two or more, unless otherwise specifically defined.

In the present disclosure, the word “exemplary” is used to mean “serving as an example, illustration or description”. Any embodiment described as exemplary in the present disclosure is not necessarily to be construed as more preferred or advantageous than other embodiments. For enabling any person skilled in the art to make and use the present disclosure, the following description is given. The following description sets forth details for the purpose of explanation. It should be understood that those of ordinary skill in the art will recognize that the present disclosure may be practiced without these specific details. In other instances, well-known structures and processes are not set forth in detail to avoid obscuring the description of the present disclosure with unnecessary details. Therefore, the present disclosure is not intended to be limited to the illustrated embodiments, but is to be accorded the widest scope consistent with the principles disclosed herein.

The existing catheters for cardiovascular, respiratory and digestive tracts are generally not bendable, or can be bent in one or two directions, and cannot be self-locked after bending, leading to poor stability, which affects the operation of an operator. On the other hand, a handle product structure is unreliable, which brings surgical risks to a patient.

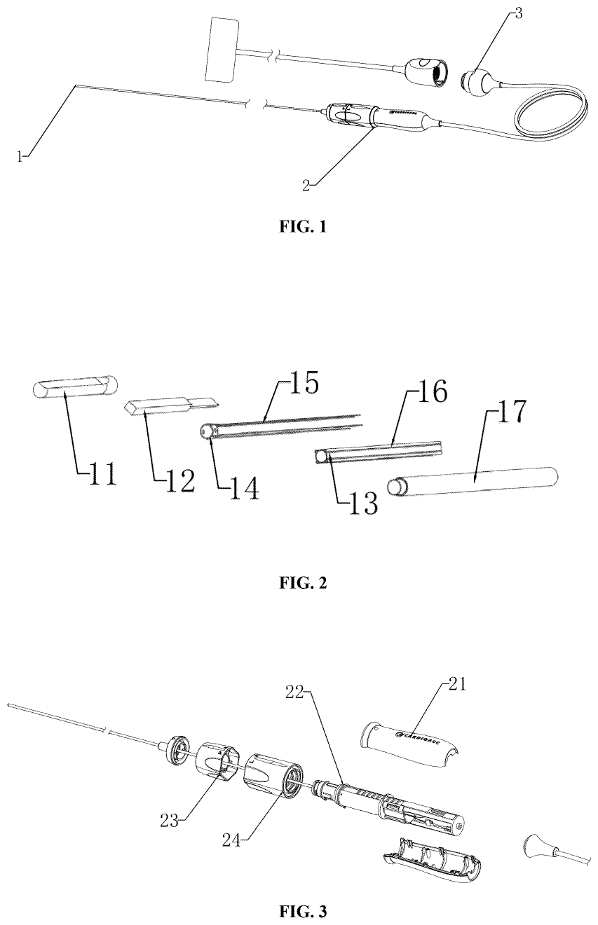

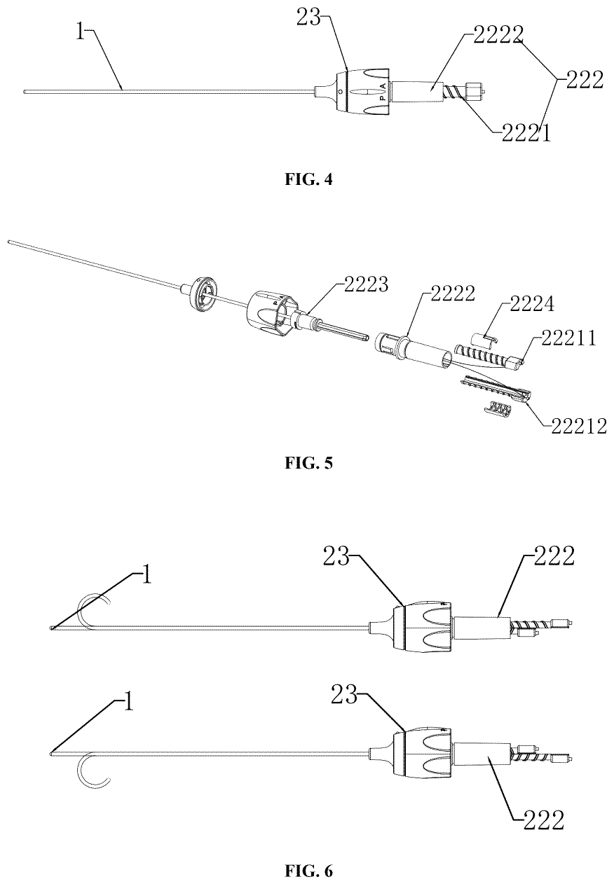

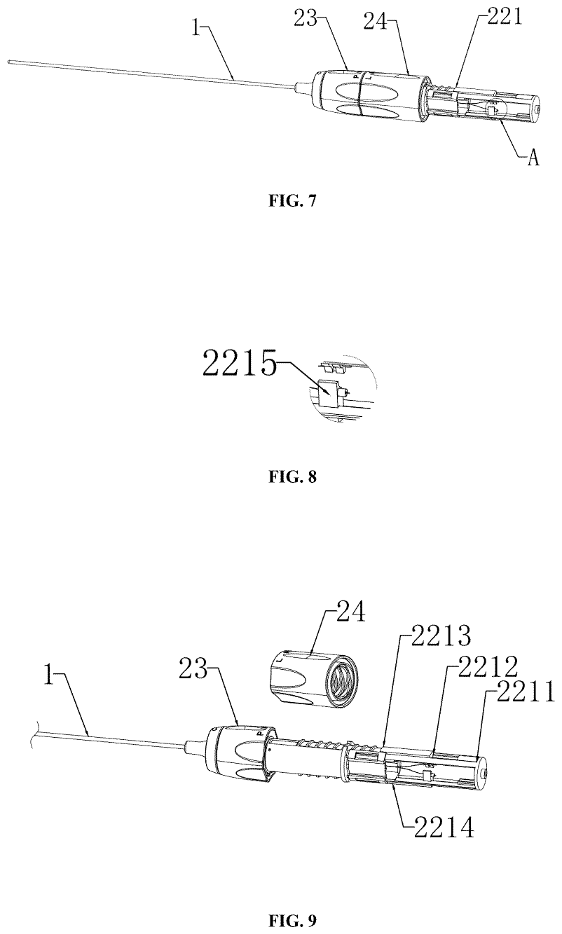

Please referring to FIG. 1 to FIG. 9, an interventional handle component with four degrees of freedom is provided, including an operating handle 2, a catheter component 1 connected to a front end of the operating handle 2, and a connector component 3 connected to a rear end of the operating handle 2. The operating handle 2 includes a holding housing 21, a transmission module 22 is accommodated in an inner cavity of the holding housing 21, and at least four traction wires which are located at different circumferential positions and extend in an axial length are arranged in the catheter component 1. A front end of each traction wire is fixedly connected to a front end of the catheter component 1, and a rear end of the traction wire is connected to the transmission module 22. The transmission module 22 includes a first transmission component 222 for controlling two of the traction wires to bend in a first plane, and a second transmission component 221 for controlling the other two traction wires to bend in a second plane. A front end, located at the holding housing 21, of the operating handle 2 is provided with a front thumbwheel 23 and a rear thumbwheel 24, the front thumbwheel 23 is in transmission connection with the first transmission component 222, and the rear thumbwheel 24 is in transmission connection with the second transmission component 221. The transmission module 22 is used to control the pulling and loosening of the traction wire, thus achieving different bending directions of the catheter component 1 embedded with the traction wire. Preferably, the front thumbwheel 23 is connected to the first transmission component 222 of the transmission module 22, and used to control a transmission structure of the first transmission component 222. The purpose of the first transmission component 222 is to control the traction wires in the first plane to bend towards two opposite directions. Similarly, the rear thumbwheel 24 is connected to the second transmission component 221 of the transmission module 22 to control the transmission structure of the second transmission component. The purpose of the second transmission component 221 is to control the traction wires in the second plane to bend in two opposite directions. The bending towards opposite directions in two different planes can achieve the bending of the catheter component 1 in at least four directions.

Preferably, the handle component provided by the present disclosure integrates a bendable interventional catheter with four degrees of freedom and an operating handle 2. The product includes a catheter component 1, an operating handle 2, and a handle-side connector. The handle-side connector is connected to a host-side connector to transmit a signal and a current. The host-side connector is connected to an external host. The catheter component 1, the operating handle 2 and the handle-side connector are all assembled, and after the inspection meets the acceptance requirements, the handle-side connector is connected to the host-side connector for signal communication under an operation of a professional doctor. After confirming that the connection is normal, the doctor puts the catheter component 1 into a blood vessel, respiratory tract and digestive tract of a patient, and rotates a corresponding rotary knob of the operating handle 2 to bend a front end of the catheter in four directions, so as to achieve complex catheter operation and carry out diagnosis and treatment.

In one embodiment, the catheter component 1 includes an outer sheath 17, an etching tube 13, and a TIP head 11. The etching tube 13 is arranged in an inner cavity of the outer sheath 17, an outer wall of the etching tube 13 is provided with at least four channels 16 for the traction wires 15 to pass through, a pull ring 14 is fixedly embedded in a front end of the outer sheath 17, and each traction wire 15 is fixedly connected to the pull ring 14. The TIP head 11 is fixedly arranged at the front end of the outer sheath 17.

Preferably, the TIP head 11 is generally made of a plastic material, such as PEBAX. The pull ring 14 generally employs a pull ring 14 made of stainless steel, while the traction wire 15 preferably employs a traction wire 15 made of stainless steel, or a traction wire 15 made of nickel-titanium wire. The etching tube 13 is a PTFE (polytetrafluoroethylene) etching tube 13 with four inner cavities for the guide wires to pass through. The outer sheath 17 is specifically of a double-layer structure, an inner layer is a double-layer structure woven by stainless steel wires, while the outer layer is made of medical plastic, generally PEBAX material.

In one embodiment, the catheter component 1 further includes a sensor 12. The sensor 12 is embedded in the TIP head 11, and a rear end connecting line passes through the inner cavity of the outer sheath 17 and extends to be connected to the connector component 3.

The sensor 2 needs to be installed in the TIP head 11 and fixed by a viscose process. The pull ring 14 and the traction wire 15 are fixed by a laser welding process, and then the pull ring 14 made of stainless steel and the traction wire 15 component made of stainless steel are inserted into the PTFE etching tube 13 with four cavities, and then fused into the outer sheath 17 by a hot melting process, thus completing the assembly of the catheter component 1.

In one embodiment, the first transmission component 222 includes a rotating shaft 2222, and a slider 2221. The rotating shaft 2222 is fixedly connected to the front thumbwheel 23, the rotating shaft 2222 is threaded to the slider 2221. The slider 2221 includes a front upper slider 22211 and a front lower slider 22212 which are separated, and two traction wires 15 located in the first plane are fixedly connected to the front upper slider 22211 and the front lower slider 22212, respectively. When the front thumbwheel 23 drives the rotating shaft 2222 to rotate, the front upper slider 22211 and the front lower slider 22212 are driven to move towards different front and rear directions, respectively.

The front thumbwheel 23 bends the front end of the catheter towards a third direction and a fourth direction, which needs the assistance of the following parts: a catheter fixing member 2223, the rotating shaft 2222, a guide block 2224, the front upper slider 22211 and the front lower slider 22212. After the parts are assembled together, when the front end of the catheter needs to be bent towards the third direction, it is only necessary to toggle the front thumbwheel 23 to rotate towards the third direction. In this case, the front lower slider 22212 pulls the traction wire 15 backwards to bend the front end of the catheter towards the third direction.

Similarly, when the front end of the catheter needs to be bent towards a fourth direction, it is only necessary to toggle the front thumbwheel 23 to rotate towards the fourth direction. In this case, the front lower slider 22212 pulls the traction wire 15 backwards to bend the front end of the catheter towards the fourth direction.

In one embodiment, the first transmission component 222 further includes a catheter fixing member 2223, and a guide block 2224. A front end of the catheter fixing member 2223 is used to fixedly connect the catheter component 1, and the catheter fixing member 2223 is installed at an inner cavity of the rotating shaft 2222. The guide block 2224 is installed at the inner cavity of the rotating shaft 2222, and an inner cavity of the guide block 2224 is provided with internal threads.

In one embodiment, surface threads of the front upper slider 22211 and the front lower slider 22212 are opposite in helical directions. The opposite thread structure can ensure that moving directions of the front upper slider 22211 and the front lower slider 22212 are opposite when driven by the threads. Therefore, when one slider pulls the traction wire 15 to tighten, the other slider can move towards an opposite direction to achieve the loosening of the traction wire 15. In this case, under the action of the traction wire 15, the catheter component 1 is bent to one side that tightens the traction wire 15.

In one embodiment, the second transmission component 221 includes a support member 2211, a surface of the support member 2211 is provided with a chute 2212, and a rear upper slider 2213 and a rear lower slider 2214 are arranged in the chute 2212. The rear thumbwheel 24 is sleeved on the rear upper slider 2213 and the rear lower slider 2214, and surfaces of the rear upper slider 2213 and the rear lower slider 2214 are provided with threads in opposite directions. When the rear thumbwheel 24 rotates, the rear upper slider 2213 and the rear lower slider 2214 move towards opposite directions.

Preferably, when a rotation direction of the catheter needs to be adjusted, the rear thumbwheel 24 is rotated to a first direction, the rear upper slider 2213 is pushed to move backwards when positive and negative threads in the thumbwheel rotate. The front end of the catheter is driven by the traction wire 15 welded to the rear upper slider 2213 of the fixing block to bend towards the first direction.

When the front end of the catheter needs to be adjusted to bend towards the second direction, the rear thumbwheel 24 is rotated towards the second direction, the rear upper slider 2213 is pushed to move backwards when the positive and negative threads inside the thumbwheel rotate. The front end of the catheter is driven by the traction wire 15 welded to the rear upper slider 2213 of the fixing block to bend towards the second direction.

In one embodiment, the surface threads of the rear upper slider 2213 and the rear lower slider 2214 are opposite in helical directions. The opposite thread structure can ensure that the moving directions of the front upper slider 22211 and the front lower slider 22212 are opposite when driven by the threads. Therefore, when one slider pulls the traction wire 15 to tighten, the other slider can move towards an opposite direction to achieve the loosening of the traction wire 15. In this case, under the action of the traction wire 15, the catheter component 1 is bent to one side that tightens the traction wire 15.

In one embodiment, the rear upper slider 2213 and the rear lower slider 2214 are provided with fixing blocks 2215, and the traction wires 15 located in the second plane are connected to the fixing blocks 2215.

In one embodiment, the first plane and the second plane are two planes perpendicular to each other. The two mutually perpendicular planes can ensure that the catheter component 1 can be bent in four mutually perpendicular directions, which can achieve more flexible catheter operation and ensure stability, thus improving the flexibility and stability of an interventional catheter operation.

In one embodiment, the front thumbwheel 23 and the rear thumbwheel 24 can rotate at an angle of 360°, and the surfaces of the front thumbwheel 23 and the rear thumbwheel 24 are provided with indicator marks for indicating the rotation direction.

In this embodiment, the front thumbwheel 23 is used to control the catheter component 1 to achieve a bending structure in an upper direction and a lower direction, and the indicator marks on the front thumbwheel 23 are P and A.

The rear thumbwheel 24 is used to control the catheter component 1 to achieve a bending structure in a left direction and a right direction, and the indicator marks on the rear thumbwheel 23 are L and R.

The present disclosure has the advantages that:

By providing a bendable intracardiac super catheter with four degrees of freedom and an operating handle, the scheme has low manufacturing cost, high reliability, and light device. More flexible catheter operation can be achieved, and stability is ensured, thus improving the flexibility and stability of an interventional catheter operation.

The above is only several specific embodiments of the present disclosure, but the present disclosure is not limited to thereto. Any changes that can be thought of by those skilled in the art should fall within the scope of protection of the present disclosure.

What is claimed is:

1. An interventional handle component with four degrees of freedom, comprising an operating handle, a catheter component connected to a front end of the operating handle, and a connector component connected to a rear end of the operating handle, wherein the operating handle comprises a holding housing, a transmission module is accommodated in an inner cavity of the holding housing, and at least four traction wires which are located at different circumferential positions and extend in an axial length are arranged in the catheter component; a front end of each traction wire is fixedly connected to a front end of the catheter component, and a rear end of the traction wire is connected to the transmission module; the transmission module comprises a first transmission component for controlling two of the traction wires to bend in a first plane, and a second transmission component for controlling the other two traction wires to bend in a second plane; a front end, located at the holding housing, of the operating handle is provided with a front thumbwheel and a rear thumbwheel, the front thumbwheel is in transmission connection with the first transmission component, and the rear thumbwheel is in transmission connection with the second transmission component.

2. The interventional handle component with four degrees of freedom according to claim 1, wherein the catheter component comprises an outer sheath, an etching tube, and a TIP head; the etching tube is arranged in an inner cavity of the outer sheath, an outer wall of the etching tube is provided with at least four channels for the traction wires to pass through, a pull ring is fixedly embedded in a front end of the outer sheath, and each traction wire is fixedly connected to the pull ring; and the TIP head is fixedly arranged at the front end of the outer sheath.

3. The interventional handle component with four degrees of freedom according to claim 2, wherein the catheter component further comprises a sensor, the sensor is embedded in the TIP head, and a rear end connecting line passes through the inner cavity of the outer sheath and extends to be connected to the connector component.

4. The interventional handle component with four degrees of freedom according to claim 1, wherein the first transmission component comprises a rotating shaft, and a slider; the rotating shaft is fixedly connected to the front thumbwheel, and the rotating shaft is threaded to the slider; the slider comprises a front upper slider and a front lower slider which are separated, and two traction wires located in the first plane are fixedly connected to the front upper slider and the front lower slider, respectively; and when the front thumbwheel drives the rotating shaft to rotate, the front upper slider and the front lower slider are driven to move in different front and rear directions, respectively.

5. The interventional handle component with four degrees of freedom according to claim 4, wherein the first transmission component further comprises a catheter fixing member, and a guide block; a front end of the catheter fixing member is used to fixedly connect the catheter component, and the catheter fixing member is installed at an inner cavity of the rotating shaft; and the guide block is installed at the inner cavity of the rotating shaft, and an inner cavity of the guide cavity is provided with internal threads.

6. The interventional handle component with four degrees of freedom according to claim 5, wherein surface threads of the front upper slider and the front lower slider have opposite helical directions.

7. The interventional handle component with four degrees of freedom according to claim 1, wherein the second transmission component comprises a support member, the surface of the support member is provided with a chute, and a rear upper slider and a rear lower slider are arranged in the chute; the rear thumbwheel is sleeved on the rear upper slider and the rear lower slider, and surfaces of the rear upper slider and the rear lower slider are provided with threads in opposite directions; and when the rear thumbwheel rotates, the rear upper slider and the rear lower slider move towards opposite directions.

8. The interventional handle component with four degrees of freedom according to claim 7, wherein the rear upper slider and the rear lower slider are provided with fixing blocks, and the traction wires located in the second plane are connected to the fixing blocks.

9. The interventional handle component with four degrees of freedom according to claim 1, wherein the first plane and the second plane are two planes perpendicular to each other.

10. The interventional handle component with four degrees of freedom according to claim 1, wherein the front and rear thumbwheels are able to rotate at an angle of 360°, and surfaces of the front and rear thumbwheels are provided with indication marks for indicating the rotation direction.
